(19) United States
(12) Patent Application Publication
(10) Pub. No.: US 2026/0242374 A1
(43) Pub. Date: Aug. 20, 2026

(54) A Process For The Preparation Of Pure Crystalline Rimegepant And Its Salts Thereof

(71) Applicant: NATCO PHARMA LIMITED, Hyderabad (IN)

(72) Inventors: Venkateswarlu TALLAPANENI, Hyderabad (IN); Kalyana Chowdary MEKA, Hyderabad (IN); Srishailam ACHINA, Hyderabad (IN); Swapna DUDDU, Hyderabad (IN); Malla Reddy SERIGUDA, Hyderabad (IN); Pulla Reddy MUDDASANI, Hyderabad (IN); Venkaiah Chowdary NANNAPANENI, Hyderabad (IN)

(21) Appl. No.: 19/161,312

(22) PCT Filed: Feb. 19, 2024
(86) PCT No.: PCT/IN2024/050173

§ 371 Date: Sep. 2, 2025

(30) Foreign Application Priority Data
Mar. 2, 2023 (IN) 202341014130

(51) Int. Cl. C07D 471/04

(52) U.S. Cl. CPC C07D 471/04

(57) ABSTRACT

The present invention relates to an improved process for the preparation of (5S,6S,9R)-5-amino-6-(2,3difluorophenyl)-6,7,8,9-tetrahydro-5H-cyclohepta[b]pyridin-9-yl-4-(2-oxo-2,3-dihydro-1H-imidazo[4,5-b]pyridin-1-yl)-1-piperidine carboxylate compound of formula-1 with desfluro impurity at less than 0.1% level and in its crystalline form which is represented by the following structural formula:

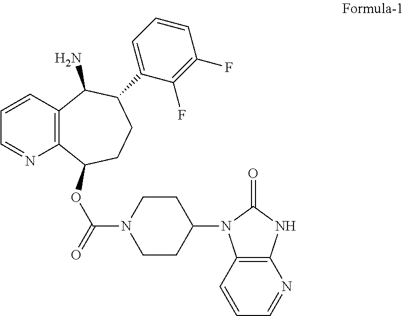 [SMILES: N[C@@H]1c2cccnc2[C@H](OC(=O)N2CCC(n3c(=O)[nH]c4ncccc43)CC2)CC[C@H]1c1cccc(F)c1F]

### FIELD OF THE INVENTION

The present invention relates to a process for the preparation of desfluoro impurity free Rimegipant ((5S,6S,9R)-5-amino-6-(2,3-difluorophenyl)-6,7,8,9-tetrahydro-5H-cyclohepta[b]pyridin-9-yl 4-(2-oxo-2,3-dihydro-1H-imidazo[4,5-b]pyridin-1-yl)-1-piperidine carboxylate) compound of formula-1 which is represented by the following structural formula:

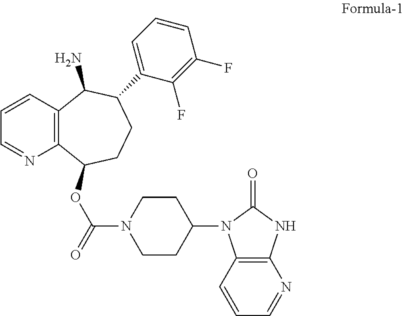 [SMILES: N[C@@H]1c2cccnc2[C@H](OC(=O)N2CCC(n3c(=O)[nH]c4ncccc43)CC2)CC[C@H]1c1cccc(F)c1F]

The present invention also relates to a process for the preparation of novel crystalline form of compound of formula-1 and its salts as well as its process for the preparation.

### BACKGROUND OF THE INVENTION

The drug compound (5S,6S,9R)-5-amino-6-(2,3difluorophenyl)-6,7,8,9-tetrahydro-5H-cyclohepta[b]pyridin-9-yl 4-(2-oxo-2,3-dihydro-1H-imidazo[4,5-b]pyridin-1-yl)-1-piperidinecarboxylate is commonly known as “Rimegepant”.

Rimegepant has been approved by U.S. Food and Drug Administration (FDA) in the form of hemisulfate sesquihydrate in February 2020 which is a medication used for the acute treatment of migraine with or without aura in adults and the preventative treatment of episodic migraine in adults. Rimegepant is marketed by Biohaven Pharmaceuticals under the brand name Nurtec ODT.

Rimegepant hemisulfate sesquihydrate is chemically known as (5S,6S,9R)-5-amino-6-(2,3-difluorophenyl)-6,7,8,9-tetrahydro-5H-cyclohepta[b]pyridin-9-yl-4-(2-oxo-2,3-dihydro-1H-imidazo[4,5-b]pyridin-1-yl)-1-piperidinecarboxylate hemisulfate sesquihydrate and is represented by the following structural formula Ia.

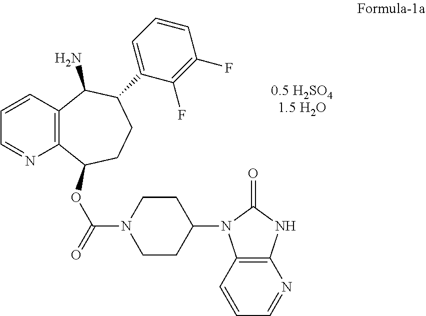 [SMILES: N[C@@H]1c2cccnc2[C@H](OC(=O)N2CCC(n3c(=O)[nH]c4ncccc43)CC2)CC[C@H]1c1cccc(F)c1F]

U.S. Pat. No. 8,314,117B2 disclosed a process for the preparation of Rimegepant. The process disclosed in US '117 involves the use of sodium azide which is highly toxic and explosive in nature and hence not suitable at commercial scale.

U.S. Pat. No. 8,669,368B2 disclosed a process for the preparation of Rimegepant. The said process involves the usage of expensive solvents for the purification of Rimegepant.

Organic Letter, 2012, 14 (18), 4938-4941 disclosed a process for the preparation of Rimegepant intermediates by asymmetric reduction methods. The said process involves the usage of metal catalyst Rh-(Rbinapine)(COD)BF4 which is expensive and not easily available in the market.

Thus, all the prior reported processes for the preparation of Rimegepant or its intermediates are having the following disadvantages such as:
The use of sodium azide which is highly toxic and explosive in nature.
Process requires flash and chromatographic purification techniques to purify the product. On higher scale production, flash and chromatographic purification techniques are time consuming and not viable as it requires huge quantity of organic solvents. The output quantity would be very low and is unviable on commercial scale to meet the market demand.
Formation of desfluoro impurity is high and no suitable conditions to control the same.
Usage of costly & hazardous reagents and reaction conditions which are not suitable for industrial production.

Therefore, there remains a need to provide a cost effective and commercially viable process for the preparation of Rimegepant.

Polymorphism, the occurrence of different crystal forms, is a property of some molecules and molecular complexes. A single molecule, may give rise to a variety of crystalline forms having distinct crystal structures and physical properties like melting point, X-ray diffraction pattern, infrared absorption fingerprint, and solid state NMR spectrum, thermogravimetric analysis (‘TGA’), and differential scanning calorimetry (‘DSC) which have been used to distinguish polymorphic forms.

As of the date, the PXRD of Rimegepant base was not characterized and disclosed in the prior-art. The present inventors have characterized and disclosed the PXRD of Rimegepant base and designated as crystalline “Form-N”.

### BRIEF DESCRIPTION OF THE INVENTION

The first aspect of the present invention is to provide a process for the preparation of Rimegepant compound of formula-1 which is free of desfluoro impurity compound of formula-1b represented as below:

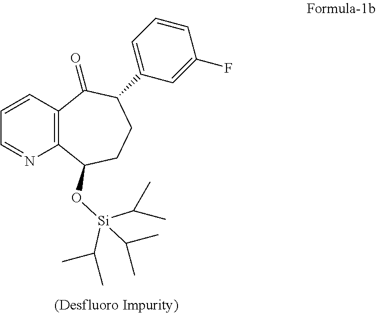 [SMILES: CC(C)[Si](O[C@@H]1CC[C@@H](c2cccc(F)c2)C(=O)c2cccnc21)(C(C)C)C(C)C]

The second aspect of the present invention is to provide a crystalline form of Rimegepant base compound of formula-1 hereinafter designated as “Form-N” and its process for the preparation.

### BRIEF DESCRIPTION OF THE DRAWINGS

FIG. 1: Illustrates the characteristic PXRD pattern of crystalline form-N of Rimegepant base (Formula-1).

### DETAILED DESCRIPTION OF THE INVENTION

The term “suitable solvent” used in the present invention refers to “hydrocarbon solvents” selected from aliphatic hydrocarbon solvents such as n-hexane, n-heptane, cyclohexane, petroleum ether and aromatic hydrocarbon solvents such as toluene, xylene and the like; “ether solvents” such as dimethyl ether, diisopropyl ether, diethyl ether, methyl tert-butyl ether, 1,2-dimethoxyethane, tetrahydrofuran, 1,4-dioxane, monoxime, dioxime and the like; “ester solvents” such as methyl acetate, ethyl acetate, isopropyl acetate, n-butyl acetate and the like; “polar-aprotic solvents such as N, N-dimethylacetamide, N,N-dimethylformamide, dimethyl sulfoxide, N-methyl pyrrolidone (NMP) and the like; “chloro solvents” such as dichloromethane/methylene chloride, dichloroethane, chloroform, carbon tetrachloride and the like; “ketone solvents” such as acetone, methyl ethyl ketone, methyl isobutyl ketone and the like; “nitrile solvents” such as acetonitrile, propionitrile, isobutyronitrile and the like; “alcoholic solvents” such as methanol, ethanol, n-propanol, isopropanol, n-butanol, isobutanol, tert.amyl alcohol, t-butanol and the like; “polar solvents” such as water or mixtures thereof.

As used herein the present invention, the term “anti-solvent” refers to a solvent which is used to precipitate the solid from a solution.

As used herein the present invention the term “suitable acid” refers to organic acids or inorganic acids. The “inorganic acid” is selected from hydrochloric acid, hydrobromic acid, sulfuric acid, nitric acid and phosphoric acid; and “organic acid” is selected from formic acid, acetic acid, propionic acid, butyric acid, valeric acid, caproic acid, trifluoroacetic acid, trifluoromethanesulfonic acid, oxalic acid, malonic acid, maleic acid, fumaric acid, malic acid, succinic acid, citric acid, aspartic acid, tartaric acid, mandelic acid, benzoic acid, salicylic acid, substituted/unsubstituted alkyl/aryl sulfonic acids such as methanesulfonic acid, ethanesulfonic acid, propanesulfonic acid, benzenesulfonic acid, p-toluenesulfonic acid, naphthalenesulfonic acid and the like or mixtures thereof.

As used herein the present invention the term “suitable base” refers to “alkali metal carbonates” such as sodium carbonate, potassium carbonate, lithium carbonate, cesium carbonate and the like; “alkali metal bicarbonates” such as sodium bicarbonate, potassium bicarbonate and the like; “alkali metal hydroxides” such as sodium hydroxide, potassium hydroxide, lithium hydroxide and the like; “alkali metal alkoxides” such as sodium methoxide, sodium ethoxide, potassium methoxide, potassium ethoxide, sodium tert-butoxide, potassium tert-butoxide, lithium tert-butoxide and the like; alkali metal hydrides such as sodium hydride, potassium hydride, lithium hydride and the like; alkali metal amides such as sodium amide, potassium amide, lithium amide and the like; Ammonia; and organic bases like dimethylamine, diethylamine, diisopropylamine, diisopropylethylamine, diisobutylamine, triethylamine, pyridine, 4-dimethylaminopyridine (DMAP), N-methyl morpholine (NMM), 2,6-lutidine, lithium diisopropylamide; organosilicon bases such as lithium hexamethyldisilazide (LiHMDS), sodium hexamethyldisilazide (NaHIMDS), potassium hexamethyldisilazide (KHMDS) or mixtures thereof.

The first aspect of the present invention is to provide an improved process for the preparation of desfluro impurity free Rimegepant compound of formula-1, comprising of:
a) Treating 7,8-dihydro-5H-cyclohepta[b]pyridine-5,9(6H)-dione compound of formula-2,

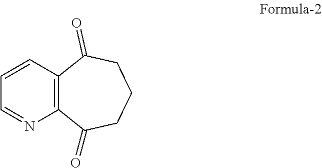 [SMILES: O=C1CCCC(=O)c2ncccc21]

with a suitable catalyst in presence of a base, organic acid and solvent followed by treating the obtained compound with IPA·HCl in a solvent to provide (9R)-9-hydroxy-6,7,8,9-tetrahydro-5H-cyclohepta[b]pyridin-5-one hydrochloride compound of formula-3,

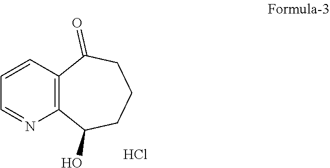 [SMILES: O=C1CCC[C@@H](O)c2ncccc21 ; Cl]

b) treating the compound of formula-3 with triisopropylsilyltrifluoromethane sulfonate in presence of triethylamine in dichloromethane to provide (9R)-9-((triisopropylsilyl)oxy)-6,7,8,9-tetrahydro-5H-cyclohepta[b]pyridin-5-one compound of formula-4,

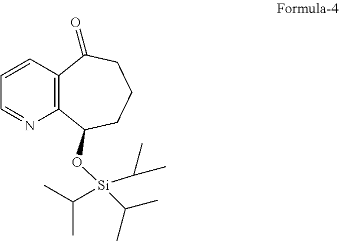 [SMILES: CC(C)[Si](O[C@@H]1CCCC(=O)c2cccnc21)(C(C)C)C(C)C]

c) reacting the compound of formula-4 with 1-bromo-2,3-difluorobenzene compound of formula-5,

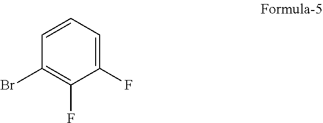

in presence of potassium phosphate base, palladium acetate catalyst and a suitable ligand in a suitable solvent to provide (6S,9R)-6-(2,3-difluoro phenyl)-9-((triisopropylsilyl)oxy)-6,7,8,9-tetrahydro-5H-cyclohepta[b]pyridine-5-one compound of formula-6,

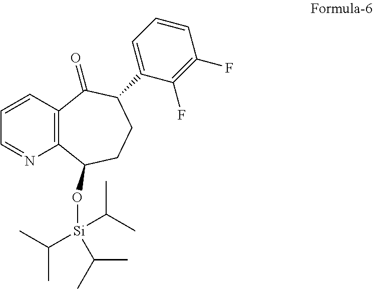 [SMILES: CC(C)[Si](O[C@@H]1CC[C@@H](c2cccc(F)c2F)C(=O)c2cccnc21)(C(C)C)C(C)C]

d) treating the compound of formula-6 with titanium tetraisopropoxide and ammonia in 1,4-dioxane followed by reduction in presence of Pd/Alumina and acetic acid to provide the compound of formula-7 in the form of a base. Treating the obtained compound with IPA-HCl in isopropanol to provide (5S,6S,9R)-6-(2,3-difluorophenyl)-9-((triisopropylsilyl)oxy)-6,7,8,9-tetrahydro-5H-cyclohepta[b]pyridine-5-amine hydrochloride,

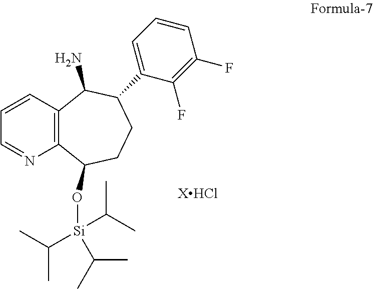

e) treating the compound of formula-7 with a mixture of isopropanol/water and neutralized with a base to provide (5S,6S,9R)-5-amino-6-(2,3-difluorophenyl)-6,7,8,9-tetrahydro-5H-cyclo hepta[b]pyridin-9-ol compound of formula-8, followed by reacting the compound of formula-8 in-situ with 1-(1-(1H-imidazole-1-carbonyl) piperidine-4-yl)-1H-imidazo[4,5-b]pyridin-2 (3H)-one compound of formula-9 in presence of potassium tert-butoxide solution in THF to provide (5S,6S,9R)-5-amino-6-(2,3-difluorophenyl)-6,7,8,9-tetrahydro-5H-cyclohepta[b]pyridin-9-yl 4-(2-oxo-2,3-dihydro-1H-imidazo[4,5-b]pyridin-1-yl)-1-piperidine-1-carboxylate compound of formula-1,
f) purifying the compound with a suitable solvent to provide pure Rimegepant compound of formula-1.

Wherein,
in step-a, c & f) the suitable solvent is selected from alcoholic solvents, polar-aprotic solvents, hydrocarbon solvents, ester solvents, ether solvents, ketone solvents, chloro solvents, nitrile solvents and polar solvents such as water or mixtures thereof.
in step-a) the suitable catalyst is RuCl(P-Cymene)(R,R)-TsDPEN;
in step-a) the suitable organic acid is formic acid;
in step-a) the suitable base is selected from organic base such as triethylamine;
in step-c) the suitable ligand is 2-Dicyclohexylphosphino-2′,6′-diisopropoxy-1,1′-biphenyl (RuPhos);
in step-e) the suitable base is selected from inorganic base such as sodium hydroxide;

The preferred embodiment of the present invention is to provide an improved process for the preparation of desfluro impurity free Rimegepant compound of formula-1, comprising of:
a) Treating 7,8-dihydro-5H-cyclohepta[b]pyridine-5,9(6H)-dione compound of formula-2 with RuCl (P-Cymene) (R, R)-Ts DPEN in presence of triethylamine & formic acid in dichloromethane followed by treating the obtained compound with IPA·HCl in isopropanol to provide (9R)-9-hydroxy-6,7,8,9-tetrahydro-5H-cyclohepta[b]pyridin-5-one hydrochloride compound of formula-3,
b) treating the compound of formula-3 with triisopropylsilyltrifluoromethane sulfonate in presence of triethylamine in dichloromethane to provide (9R)-9-((triisopropylsilyl)oxy)-6,7,8,9-tetrahydro-5H-cyclohepta[b]pyridin-5-one compound of formula-4,
c) reacting the compound of formula-4 with 1-bromo-2,3-difluorobenzene compound of formula-5 in presence of tribasic potassium phosphate base, palladium acetate catalyst and RuPhos ligand in tert-amyl alcohol to provide (6S,9R)-6-(2,3-difluorophenyl)-9-((triisopropylsilyl)oxy)-6,7,8,9-tetrahydro-5H-cyclohepta[b]pyridin-5-one compound of formula-6,
d) treating the compound of formula-6 with titanium tetraisopropoxide and ammonia in 1,4-dioxane followed by reduction in presence of Pd/Alumina and acetic acid to provide the free base compound of formula-7. Treating the obtained compound with IPA-HCl in isopropanol to provide (5S,6S,9R)-6-(2,3-difluorophenyl)-9-((triisopropylsilyl)oxy)-6,7,8,9-tetrahydro-5H-cyclohepta[b]pyridine-5-amine hydrochloride,
e) treating the compound of formula-7 with a mixture of isopropanol/water and neutralized with sodium hydroxide to provide (5S,6S,9R)-5-amino-6-(2,3-difluorophenyl)-6,7,8,9-tetrahydro-5H-cyclo hepta[b]pyridin-9-ol compound of formula-8, followed by reacting the compound of formula-8 in-situ with 1-(1-(1H-imidazole-1-carbonyl) piperidine-4-yl)-1H-imidazo[4,5-b]pyridin-2-(3H)-one compound of formula-9 in presence of potassium tert-butoxide solution in THF to provide (5S,6S,9R)-5-amino-6-(2,3-difluorophenyl)-6,7,8,9-tetrahydro-5H-cyclohepta[b]pyridin-9-yl 4-(2-oxo-2,3-dihydro-1H-imidazo[4,5-b]pyridin-1-yl)-1-piperidine-1-carboxylate compound of formula-1,
f) purifying the compound with methanol to provide pure Rimegepant compound of formula-1.

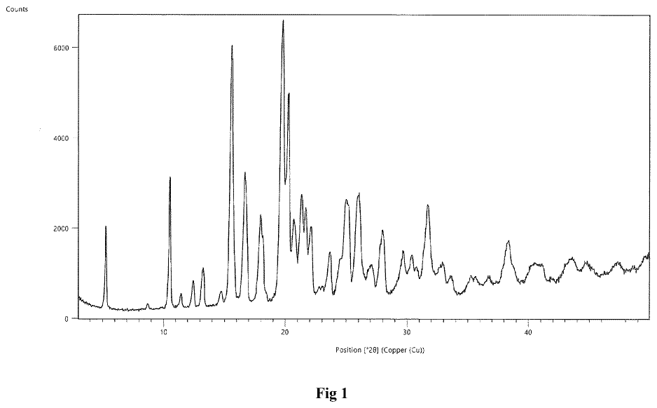

The second aspect of the present invention is to provide a crystalline form-N of Rimegepant base compound of formula-1, which is characterized by:
i) Its powder X-ray diffractogram having peaks at about 5.2, 10.5, 15.5 16.6, 17.9, 19.8, 20.2, 24.9, 26.1 and 31.7±0.2 degrees 2-theta.
ii) Its powder X-ray diffraction pattern as shown in FIG. 1.

Further, the present invention also provides a process for the preparation of crystalline Form-N of Rimegepant base compound of formula-1, comprising of:
a) Treating the compound of formula-7 with a mixture of isopropanol/water and neutralized with a base to provide (5S,6S,9R)-5-amino-6-(2,3-difluorophenyl)-6,7,8,9-tetrahydro-5H-cyclohepta[b]pyridin-9-ol compound of formula-8,
b) reacting the compound of formula-8 in-situ with 1-(1-(1H-imidazole-1-carbonyl) piperidine-4-yl)-1H-imidazo[4,5-b]pyridin-2 (3H)-one compound of formula-9 in presence of potassium tert-butoxide solution in THF to provide (5S,6S,9R)-5-amino-6-(2,3-difluorophenyl)-6,7,8,9-tetrahydro-5H-cyclohepta[b]pyridin-9-yl 4-(2-oxo-2,3-dihydro-1H-imidazo[4,5-b]pyridin-1-yl)-1-piperidine-1-carboxylate compound of formula-1,
c) purifying the compound with a suitable solvent to provide crystalline Form-N of Rimegepant compound of formula-1.

Wherein,
in step-a) the suitable base is selected from inorganic base such as sodium hydroxide;
in step-c) the suitable solvent is selected from alcohol solvent;

The preferred embodiment of the present invention provides a process for the preparation of crystalline Form-N of Rimegepant base compound of formula-1, comprising of:
a) Treating the compound of formula-7 with a mixture of isopropanol/water and neutralized with sodium hydroxide to provide (5S,6S,9R)-5-amino-6-(2,3-difluorophenyl)-6,7,8,9-tetrahydro-5H-cyclohepta[b]pyridin-9-ol compound of formula-8,
b) reacting the compound of formula-8 in-situ with 1-(1-(1H-imidazole-1-carbonyl) piperidine-4-yl)-1H-imidazo[4,5-b]pyridin-2 (3H)-one compound of formula-9 in presence of potassium tert-butoxide solution in THF to provide (5S,6S,9R)-5-amino-6-(2,3-difluorophenyl)-6,7,8,9-tetrahydro-5H-cyclohepta[b]pyridin-9-yl 4-(2-oxo-2,3-dihydro-1H-imidazo[4,5-b]pyridin-1-yl)-1-piperidine-1-carboxylate compound of formula-1,
c) purifying the compound with methanol to provide crystalline Form-N of Rimegepant compound of formula-1.

The 7,8-dihydro-5H-cyclohepta[b]pyridine-5,9(6H)-dione compound of formula-2, 1-bromo-2,3-difluorobenzene and compound of formula-5 & 1-(1-(1H-imidazole-1-carbonyl) piperidine-4-yl)-1H-imidazo[4,5-b]pyridin-2 (3H)-one compound of formula-9 are prepared from the processes known in the art.

The crystalline Form-N of Rimegepant prepared from the present invention is useful in the preparation of Rimegepant hemisulfate sesquihydrate of formula Ia.

### PXRD Method of Analysis:

PXRD analysis of the crystalline form of Rimegepant base was carried out using Panlytical Expert Pro DY3248 X-ray powder diffractometer using Cu-Ka radiation of 10 wavelength 1.5406 A° and at continuous scan speed of 0.03°/min.

The process for the preparation of Rimegepant compound of formula-1 is schematically represented as below:

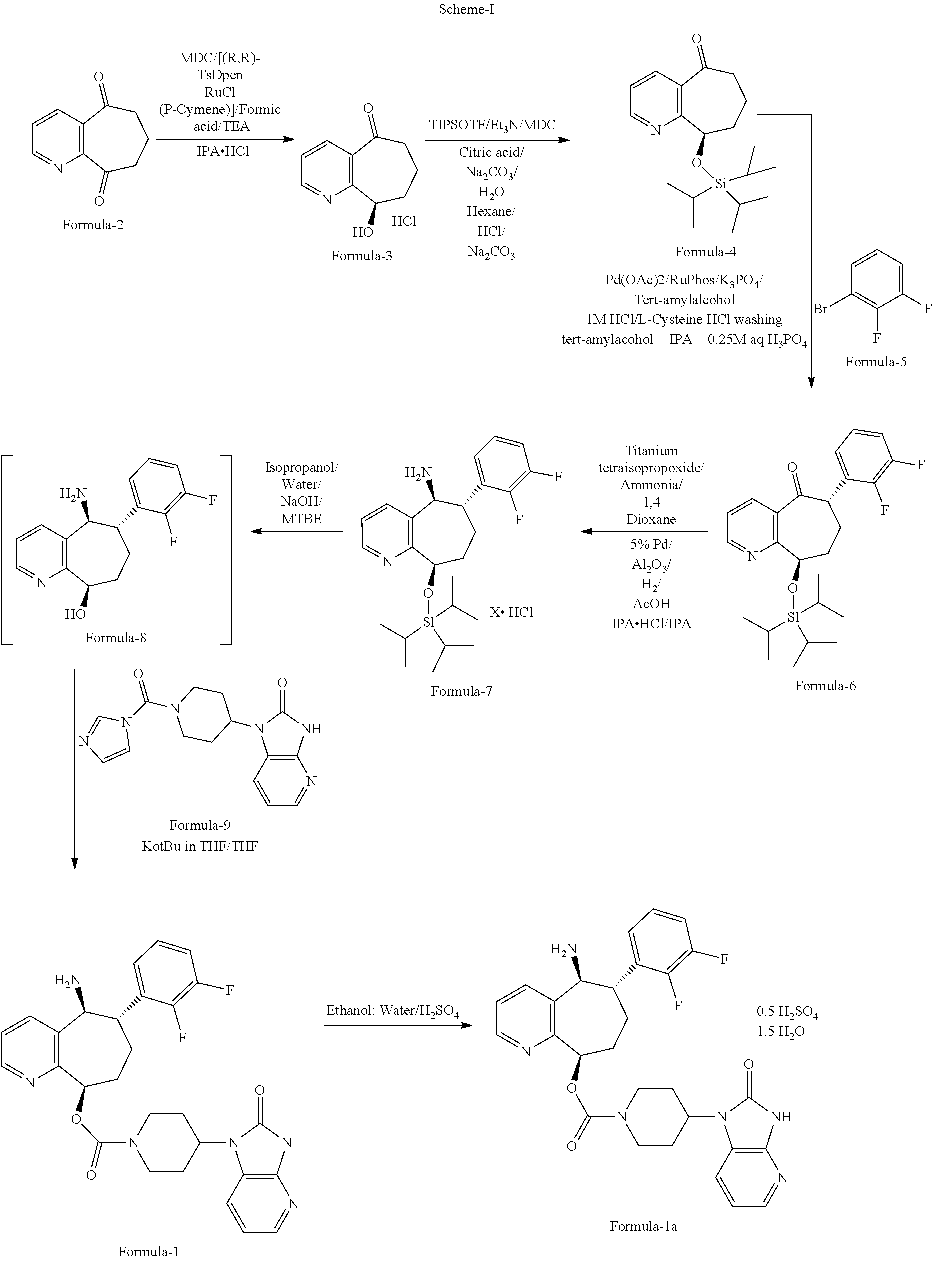

### Advantages of the Present Invention

The process of the present invention does not require chromatographic purification techniques like flash chromatography, column chromatography, preparative HPLC or preparative TLC in any stage of the process.
Present process is capable of controlling desfluoro impurity at less than 0.15% level.
The present invention avoids the usage of expensive reagents & toxic reagents and solvents.
The present invention involves the use of RuCl(Cymene)(R,R)-TsDPEN) which is easily available and more cheaper than the Rh-(Rbinapine)(COD)BF4 catalyst.
The present invention involves the usage of acetic acid to avoid the inconsistency in yields of compound of formula-7.
The present invention involves the usage of low-cost reagents and solvents, which decrease the cost of production and suitable for the commercial scale process.
The process of the present invention provides crystalline Rimegepant with high yield and purity.

The best mode of carrying out the present invention was illustrated by the below mentioned examples. These examples provide as illustration only and hence should not be construed as limitation of the scope of the invention.

### EXAMPLES

### Example-1: Preparation of (9R)-9-((triisopropylsilyl)oxy)-6,7,8,9-tetrahydro-5H-cyclohepta[b]pyridin-5-one (Formula-4)

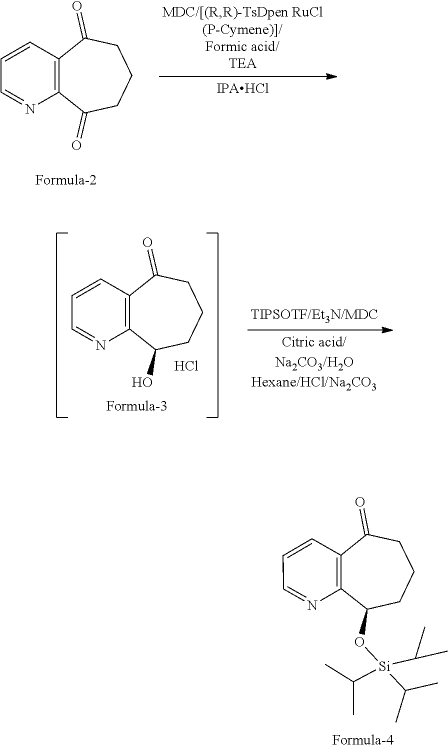

### Step-a): Preparation of (9R)-9-hydroxy-6,7,8,9-tetrahydro-5H-cyclohepta[b]pyridin-5-one hydrochloride (Formula-3)

Into a 4NRB flask, 7,8-dihydro-5H-cyclohepta[b]pyridine-5,9(6H)-dione (Formula-2; 130 g, 0.742 moles) and dichloromethane (1300 mL) were charged at 25-30° C. After stirring for 5 min, formic acid (41.0 g, 0.891 moles) was added followed by trimethylamine (90.1 g, 0.891 moles) at 25-30° C. Stirred the reaction mixture for 10 min. The reaction mixture was treated with catalyst RuCl (P-Cymene) (R, R)-Ts DPEN (1.4 g, 0.3 mole %) at 30-33° C. for 3-4 h, cooled to 25-30° C. The reaction mixture was washed with 5% aqueous citric acid solution (650 mL) followed by 10% aqueous sodium bicarbonate solution, stirred for 5 min then dichloromethane layer separated and was dried over sodium sulphate. The solvent was removed under reduced pressure resulting in a light brown oil which was dissolved in isopropanol (390 mL). The isopropanol solution was added slowly to IPA-HCl (325 mL) at 25-30° C. The resulting slurry was stirred for 1 h at 25-30° C. and then filtered and washed with isopropanol (65 mL) suck dried for 25-30 min. The wet cake was transferred into a RB flask and given hot slurry washed with isopropanol (300 mL), then dried at 40° C. under vacuum to produce (9R)-9-hydroxy-6,7,8,9-tetrahydro-5H-cyclohepta[b]pyridin-5-one hydrochloride compound of formula-3 as a light tan solid (72% yield, HPLC purity>99.0%, chiral purity 99.2% ee).

### Step-b): Preparation of (9R)-9-((triisopropylsilyl)oxy)-6,7,8,9-tetrahydro-5H-cyclohepta[b]pyridin-5-one (Formula-4)

Into a 1 L 4N RB flask, (9R)-9-hydroxy-6,7,8,9-tetrahydro-5H-cyclohepta[b]pyridin-5-one hydrochloride (Formula-3; 115.0 g, 0.538 moles), water (145.0 mL) and dichloromethane (720 mL) were charged at 25-30° C. After stirring for 10 min, reaction mass pH adjusted to 8.5-9.0 by adding sodium carbonate solution, stirred for 5 min then dichloromethane layer separated and was dried over sodium sulphate. The solvent was removed under reduced pressure resulting crude which was dissolved in 290 mL of dichloromethane and added triethylamine (108.9 g, 1.076 moles) and stirred for 10 min at 25-30° C. The reaction mixture was cooled to 0-5° C. and added triisopropylsilyltrifluoro methanesulfonate (186.3 g, 0.618 moles) keeping the internal temperature <5° C. After stirring for 60 min, saturated ammonium chloride solution (150 mL) was added and stirred for 10 min, then dichloromethane layer was separated. The solvent was removed under reduced pressure resulting in a light brown oil which was dissolved in hexane (1150 mL) and separated the oily layer. The hexane solution was added to hydrochloric acid (350 mL) at 25-30° C. and stirred for 10 min then hydrochloric acid layer was separated. The hydrochloric acid layer pH was adjusted to 5.5±0.2 by adding 30% aq. sodium carbonate solution. The resulting slurry was stirred for 20-30 min at 25-30° C. and then filtered. The cake was slurry washed with water (450 mL), then dried at 40° C. under vacuum to produce (9R)-9-((triisopropylsilyl)oxy)-6,7,8,9-tetrahydro-5H-cyclohepta[b]pyridin-5-one compound of formula-4 as an off white colour solid (88% yield, HPLC purity>99.0%, chiral purity 99.0% ee).

### Example-2: Preparation of (6S,9R)-6-(2,3-difluorophenyl)-9-((triisopropylsilyl)oxy)-6,7,8,9-tetrahydro-5H-cyclohepta[b]pyridin-5-one (Formula-6)

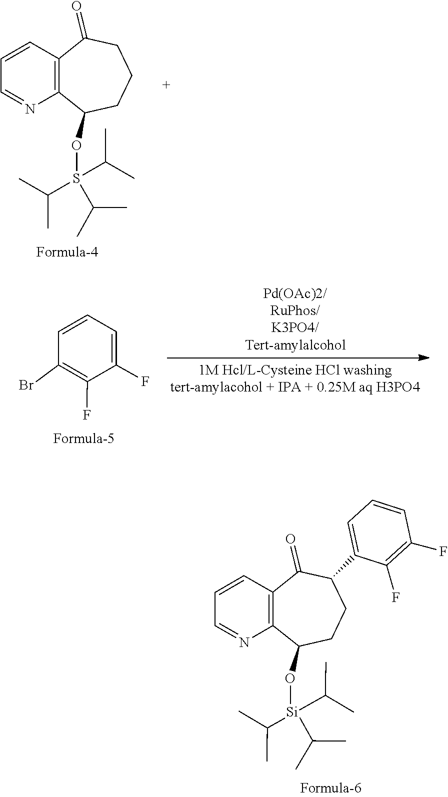 [SMILES: CC(C)S(O[C@@H]1CCCC(=O)c2cccnc21)(C(C)C)C(C)C ; Fc1cccc(Br)c1F ; CC(C)[Si](O[C@@H]1CC[C@@H](c2cccc(F)c2F)C(=O)c2cccnc21)(C(C)C)C(C)C]

Into a 1 L 4NRB flask equipped with an overhead stirrer was charged with (9R)-9-((triisopropylsilyl)oxy)-6,7,8,9-tetrahydro-5H-cyclohepta[b]pyridin-5-one (Formula-4; 120.0 g, 0.360 moles), tert-amyl alcohol (1200 mL), potassium phosphate tribasic anhydrous (306.0 g, 1.441 moles), palladium acetate (2.4 g, 0.011 moles), RuPhos catalyst (10.1 g, 0.0216 moles), and 1-bromo-2,3-difluorobenzene (Formula-5; 83.5 g, 0.432 moles) under a nitrogen atmosphere. The resulting suspension was heated to 95-100° C. and maintained at >98° C. until <5% of compound of formula-4 monitored by HPLC analysis. The mixture was cooled to 25-30° C. followed by slow addition of aq HCl (2.4 L) to the mixture while maintaining the reaction mass temperature below 30° C. The resulting biphasic mixture was stirred for 15 min at 25-30° C. and then tert-amyl alcohol layer was separated. The rich organic layer was treated with an 5% aqueous solution of L-cysteine HCl (1200 mL). The resulting biphasic mixture was heated to 50-55° C. for 2 h followed by cooling to 25-30° C. Then organic layer was separated and the rich organic layer was filtered on the Buchner funnel to remove undissolved matter. The solvent was removed under reduced pressure resulting in a dark brown oil which was dissolved in (220 ml of aqueous H₃PO₄+175 ml of isopropanol+87 ml of tert-amyl alcohol). The resulting slurry was stirred at 40° C. for 1 h, followed by cooling to 25-30° C. and stirred for 1 h, and then further cooled to 0-5° C. stirred for 2 h, and isolated by filtration. The wet cake was washed with aq H₃PO₄/isopropanol/tert-amyl alcohol, and suck dried. The wet solid material was dissolved in hexane (1200 mL) and filtered to remove undissolved matter. The solvent was removed under reduced pressure to yield solid and then charged water (1200 mL), stirred and filtered, and dried at 40° C. under vacuum to produce (6S,9R)-6-(2,3-difluorophenyl)-9-((triisopropylsilyl)oxy)-6,7,8,9-tetrahydro-5H-cyclohepta[b]pyridin-5-one compound of formula-6 as a light brown solid mixture of diastereomers (102 g, 63% yield, HPLC purity>98.0%.

### Example-3: Preparation of (5S,6S,9R)-6-(2,3-difluorophenyl)-9-((triisopropylsilyl)oxy)-6,7,8,9-tetrahydro-5H-cyclohepta[b]pyridine-5-amine hydrochloride (Formula-7)

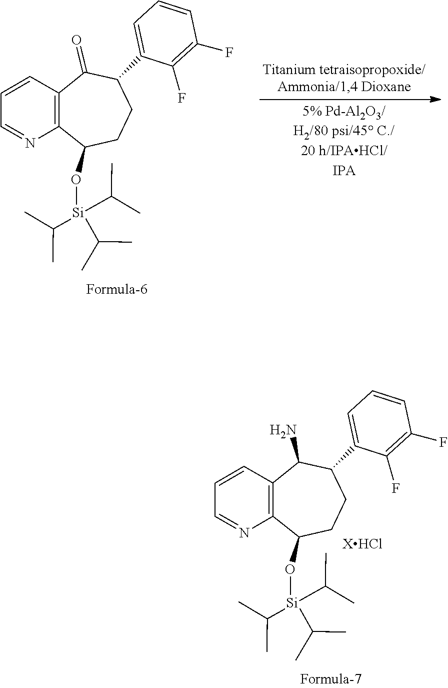

A 1 L autoclave reactor was charged with (6S, 9R)-6-(2,3-difluorophenyl)-9-(triisopropylsilyloxy)-6,7,8,9-tetrahydro-5H-cycloheptabpyridin-5-one (Formula-6; 50.00 g, 1.122 mol, Formula-6) and 1,4-dioxane (500 ml) and titanium tetraisopropoxide (80.0 g, 2.5 eq). The reactor was purged three times with nitrogen and three times with ammonia. After the purge cycle was completed, the reactor was pressurized with ammonia to 80 psi. The reaction mixture was heated to 50° C. and stirred for 18 h at 80 psi and 50° C. The progress of the reaction was monitored by HPLC (SM<5.0%) The mixture was then cooled to 20° C. The reactor was purged three times with nitrogen and three times with hydrogen. After the purged cycle was completed, then acetic acid (13.5 g, 2.5 eq) and 5% Pd/alumina (7.5 g, 15% w/w loading) was charged to the autoclave reactor. The reactor was pressurized with hydrogen to 80 psi and the mixture was heated to 45° C. and stirred for 20 h at 80 psi H₂ gas. The progress of the reaction was monitored by HPLC. The mixture was then cooled to 20° C. and filtered, then transferred to a 3-necked flask. To the reaction mixture, 50 mL of ammonia solution was added dropwise, which resulted in a slurry. The resulting slurry was stirred for 30 min then filtered, then the titanium dioxide cake was washed with 1,4-dioxane (400 mL). The filtrate was collected, and the solvent was removed. The resulting oil was dissolved in isopropanol (750 mL). To the isopropanol solution, IPA-HCl (100.0 mL) was added dropwise resulting in a thick slurry. The slurry was heated to 40-45° C. and stirred for 45-60 min. Then cooled to 20-22° C. and filtered to afford a white solid. The wet solid was slurry washed with isopropanol (250 mL) and then dried at 50° C. under vacuum to produce (5S,6S,9R)-6-(2,3-difluorophenyl)-9-((triisopropylsilyl)oxy)-6,7,8,9-tetrahydro-5H-cyclohepta[b]pyridine-5-amine hydrochloride (Formula-7) as an off-white color solid (31.2 g, 53.5% yield, HPLC purity>98.0%). Desfluoro impurity: Less than 0.15% by HPLC.

### Example-4: Preparation of (5S,6S,9R)-5-amino-6-(2,3-difluorophenyl)-6,7,8,9-tetrahydro-5H-cyclohepta[b]pyridin-9-yl 4-(2-oxo-2,3-dihydro-1H-imidazo[4,5-b]pyridin-1-yl)-1-piperidine-1-carboxylate (Formula-1)

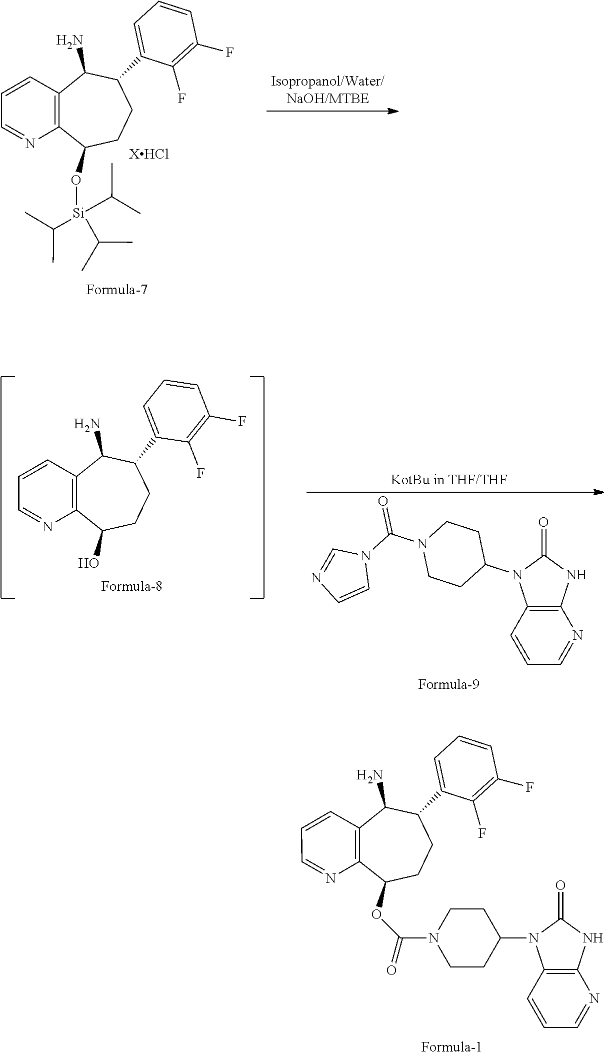

A 4NRB flask was charged with (5S,6S,9R)-6-(2,3-difluorophenyl)-9-((triisopropyl silyl)oxy)-6,7,8,9-tetrahydro-5H-cyclohepta[b]pyridine-5-amine hydrochloride (Formula-7) (28.0 g) and a solution of isopropanol (84.0 mL): water (28.0 mL). The mixture was heated to 75-80° C. and then stirred for 5-6 h. The solvent was removed under reduced pressure at 50° C. The resulting crude was dissolved in water (140.0 mL), then the water layer was washed two times with methyl tertiary-butyl ether (84.0 mL) and the methyl tertiary-butyl ether layer was separated and discarded. To the aqueous layer, sodium hydroxide (5.82 g) and methyl tertiary-butyl ether (84.0 mL) was added at 25-30° C. and then stirred for 20-30 min. The organic layer was separated and dried over on sodium sulfate. The solvent was removed under reduced pressure resulting in crude which was dissolved in tetrahydrofuran (84.0 mL) and 1-(1-(1H-imidazole-1-carbonyl) piperidin-4-yl)-1H-imidazo [4,5-bipyridin-2 (3H)-one (Formula-9) (20.2 g) was added at 25-30° C. After stirring for 10 min, the tetrahydrofuran solvent was strip-off with three times tetrahydrofuran. The resulting crude was again dissolved in tetrahydrofuran (140.0 mL), then cooled to 0-5° C. At 0° C. was added, 136 mL of 1M potassium tert-butoxide solution in THF. The thick slurry was stirred for 1 h. The progress of the reaction was monitored by HPLC (SM<2.0%) and then the reaction was quenched with the addition of 70 mL of 20 wt % aqueous sodium chloride and 70 mL of 20 wt % aqueous citric acid. The layers were allowed to separate and the organic-rich layer was retained. The organic layer was washed with 20 wt % aqueous sodium chloride. The solvent was removed under reduced pressure at 50° C. to afford an oil, which was crystallized from methanol to afford an off-white solid of (5S,6S,9R)-5-amino-6-(2,3-difluorophenyl)-6,7,8,9-tetrahydro-5H-cyclohepta[b]pyridin-9-yl 4-(2-oxo-2,3-dihydro-1H-imidazo[4,5-b]pyridin-1-yl)-1-piperidine-1-carboxylate compound of formula-1. (23.0 g, 78.3% yield, HPLC Purity >99.0%).

### Example-5: Preparation of (5S,6S,9R)-5-amino-6-(2,3difluorophenyl)-6,7,8,9-tetrahydro-5H-cyclohepta[b]pyridin-9-yl 4-(2-oxo-2,3-dihydro-1H-imidazo[4,5-b]pyridin-1-yl)-1-piperidine-1-carboxylate hemisulfate sesquihydrate (Formula-1a)

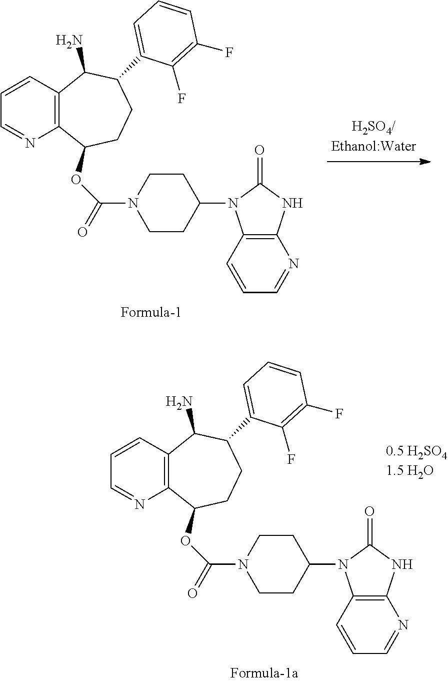 [SMILES: N[C@@H]1c2cccnc2[C@H](OC(=O)N2CCC(n3c(=O)[nH]c4ncccc43)CC2)CC[C@H]1c1cccc(F)c1F ; N[C@@H]1c2cccnc2[C@H](OC(=O)N2CCC(n3c(=O)[nH]c4ncccc43)CC2)CC[C@H]1c1cccc(F)c1F]

A 4NRB flask was charged (5S,6S,9R)-5-amino-6-(2,3-difluorophenyl)-6,7,8,9-tetrahydro-5H-cyclohepta[b]pyridin-9-yl 4-(2-oxo-2,3-dihydro-1H-imidazo[4,5-b]pyridin-1-yl)-1-piperidine-1-carboxylate (Formula-1) (20.0 g), ethanol (255 mL), and water 85.0 mL (3:1) at 25-30° C. After stirring for 10 min, the reaction mass was heated to 70±3° C. Separately 160 mL of dilute sulphuric acid solution prepared with 1.8 g H₂SO₄ (0.5 equiv) was dissolved in ethanol (120 mL) and water (40 mL) (3:1) at 25-30° C. This diluted sulphuric acid solution was added to the above reaction mass over 2 h at 70±3° C. The resulting slurry was stirred at 70° C. for 1 h and cooled to 20-25° C. over 90 min. The slurry was stirred at 20-25° C. for 24 h. The slurry was filtered. The wet cake was washed with 20 mL mixture of ethanol:water solution (3:1) and dried at 40-45° C. in a vacuum oven for 10-12 h to afford Rimegepant sulfate of formula-1a (19.5 g, 85% yield, HPLC purity >99.7%, and all impurities <0.15% as an off-white crystalline solid of polymorphic form H1.5-1.

We claim:

1. An improved process for the preparation of desfluro impurity free Rimegepant compound of formula-1, comprising of:
a) Treating 7,8-dihydro-5H-cyclohepta[b]pyridine-5,9(6H)-dione compound of formula-2, 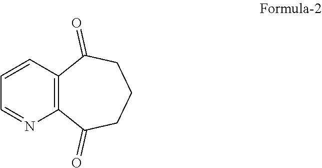 [SMILES: O=C1CCCC(=O)c2ncccc21]
with a suitable catalyst in presence of a base, organic acid and solvent followed by treating the obtained compound with IPA·HCl in a solvent to provide (9R)-9-hydroxy-6,7,8,9-tetrahydro-5H-cyclohepta[b]pyridin-5-one hydrochloride compound of formula-3, 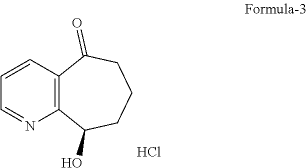 [SMILES: O=C1CCC[C@@H](O)c2ncccc21 ; Cl]
b) treating the compound of formula-3 with triisopropylsilyltrifluoromethane sulfonate in presence of triethylamine in dichloromethane to provide (9R)-9-((triisopropylsilyl)oxy)-6,7,8,9-tetrahydro-5H-cyclohepta[b]pyridin-5-one compound of formula-4, 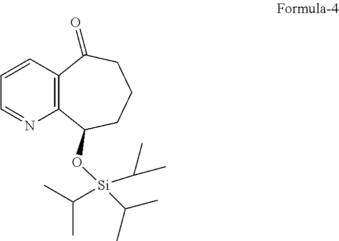 [SMILES: CC(C)[Si](O[C@@H]1CCCC(=O)c2cccnc21)(C(C)C)C(C)C]
c) reacting the compound of formula-4 with 1-bromo-2,3-difluorobenzene compound of formula-5 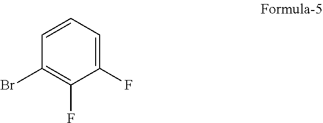
in presence of potassium phosphate base, palladium acetate catalyst and a suitable ligand in a suitable solvent to provide (6S,9R)-6-(2,3-difluoro phenyl)-9-((triisopropylsilyl)oxy)-6,7,8,9-tetrahydro-5H-cyclohepta[b]pyridine-5-one compound of formula-6, 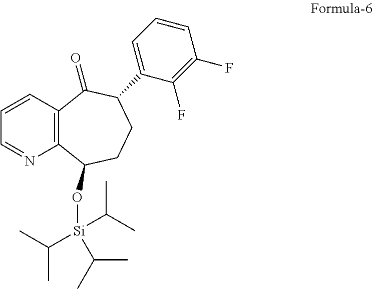 [SMILES: CC(C)[Si](O[C@@H]1CC[C@@H](c2cccc(F)c2F)C(=O)c2cccnc21)(C(C)C)C(C)C]
d) treating the compound of formula-6 with titanium tetraisopropoxide and ammonia in 1,4-dioxane followed by reduction in presence of Pd/Alumina and acetic acid to provide the compound of formula-7 in the form of a base. Treating the obtained compound with IPA-HCl in isopropanol to provide (5S,6S,9R)-6-(2,3-difluorophenyl)-9-((triisopropylsilyl)oxy)-6,7,8,9-tetrahydro-5H-cyclohepta[b]pyridine-5-amine hydrochloride, 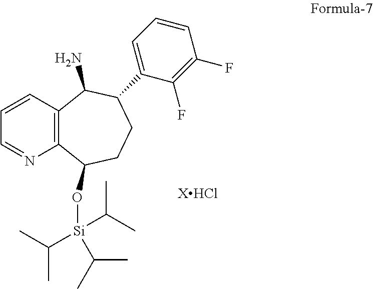
e) treating the compound of formula-7 with a mixture of isopropanol/water and neutralized with a base to provide (5S,6S,9R)-5-amino-6-(2,3-difluorophenyl)-6,7,8,9-tetrahydro-5H-cyclohepta[b]pyridin-9-ol compound of formula-8, followed by reacting the compound of formula-8 in-situ with 1-(1-(1H-imidazole-1-carbonyl) piperidine-4-yl)-1H-imidazo[4,5-b]pyridin-2 (3H)-one compound of formula-9 in presence of potassium tert-butoxide solution in THF to provide (5S,6S,9R)-5-amino-6-(2,3-difluorophenyl)-6,7,8,9-tetrahydro-5H-cyclohepta[b]pyridin-9-yl 4-(2-oxo-2,3-dihydro-1H-imidazo[4,5-b]pyridin-1-yl)-1-piperidine-1-carboxylate compound of formula-1,
f) purifying the compound with a suitable solvent to provide pure Rimegepant compound of formula-1.

2. The process as claimed in claim-1, wherein,
in step-a, c & f) the suitable solvent is selected from alcoholic solvents, polar-aprotic solvents, hydrocarbon solvents, ester solvents, ether solvents, ketone solvents, chloro solvents, nitrile solvents and polar solvents such as water or mixtures thereof,
in step-a) the suitable catalyst is RuCl(P-Cymene)(R,R)-TsDPEN;
in step-a) the suitable organic acid is formic acid;
in step-a) the suitable base is selected from organic base such as triethylamine;
in step-c) the suitable ligand is 2-Dicyclohexylphosphino-2′,6′-diisopropoxy-1,1′-biphenyl (RuPhos);
in step-e) the suitable base is selected from inorganic base such as sodium hydroxide;

3. The process as claimed in claim-1, comprising of:
a) Treating 7,8-dihydro-5H-cyclohepta[b]pyridine-5,9(6H)-dione compound of formula-2 with RuCl (P-Cymene) (R, R)-Ts DPEN in presence of triethylamine & formic acid in dichloromethane followed by treating the obtained compound with IPA·HCl in isopropanol to provide (9R)-9-hydroxy-6,7,8,9-tetrahydro-5H-cyclohepta[b]pyridin-5-one hydrochloride compound of formula-3,
b) treating the compound of formula-3 with triisopropylsilyltrifluoromethane sulfonate in presence of triethylamine in dichloromethane to provide (9R)-9-((triisopropylsilyl)oxy)-6,7,8,9-tetrahydro-5H-cyclohepta[b]pyridin-5-one compound of formula-4,
c) reacting the compound of formula-4 with 1-bromo-2,3-difluorobenzene compound of formula-5 in presence of tribasic potassium phosphate base, palladium acetate catalyst and RuPhos ligand in tert-amyl alcohol to provide (6S,9R)-6-(2,3-difluorophenyl)-9-((triisopropylsilyl)oxy)-6,7,8,9-tetrahydro-5H-cyclohepta[b]pyridin-5-one compound of formula-6,
d) treating the compound of formula-6 with titanium tetraisopropoxide and ammonia in 1,4-dioxane followed by reduction in presence of Pd/Alumina and acetic acid to provide the free base compound of formula-7. Treating the obtained compound with IPA-HCl in isopropanol to (5S,6S,9R)-6-(2,3-difluorophenyl)-9-((triisopropylsilyl)oxy)-6,7,8,9-tetrahydro-5H-cyclohepta[b]pyridine-5-amine hydrochloride,
e) treating the compound of formula-7 with a mixture of isopropanol/water and neutralized with sodium hydroxide to provide (5S,6S,9R)-5-amino-6-(2,3-difluorophenyl)-6,7,8,9-tetrahydro-5H-cyclohepta[b]pyridin-9-ol compound of formula-8, followed by reacting the compound of formula-8 in-situ with 1-(1-(1H-imidazole-1-carbonyl) piperidine-4-yl)-1H-imidazo[4,5-b]pyridin-2 (3H)-one compound of formula-9 in presence of potassium tert-butoxide solution in THF to provide (5S,6S,9R)-5-amino-6-(2,3-difluorophenyl)-6,7,8,9-tetrahydro-5H-cyclohepta[b]pyridin-9-yl 4-(2-oxo-2,3-dihydro-1H-imidazo[4,5-b]pyridin-1-yl)-1-piperidine-1-carboxylate compound of formula-1,
f) purifying the compound with methanol to provide pure Rimegepant compound of formula-1.

4. The process as claimed in claim-1, wherein the (9R)-9-hydroxy-6,7,8,9-tetrahydro-5H-cyclohepta[b]pyridin-5-one hydrochloride compound of formula-3 is prepared by treating 7,8-dihydro-5H-cyclohepta[b]pyridine-5,9(6H)-dione compound of formula-2, with a suitable catalyst in presence of a base, organic acid and solvent followed by treating the obtained compound with IPA·HCl in a solvent to provide compound of formula-3.

5. The process as claimed in claim-4, wherein, the suitable catalyst is RuCl(P-Cymene)(R,R)-TsDPEN; the suitable organic acid is formic acid; the suitable base is selected from organic base such as triethylamine; the suitable solvent is chloro solvent or alcohol solvent.

6. The process as claimed in claim-1, wherein the (9R)-9-((triisopropylsilyl)oxy)-6,7,8,9-tetrahydro-5H-cyclohepta[b]pyridin-5-one compound of formula-4 is prepared by treating the compound of formula-3 with triisopropylsilyl trifluoromethane sulfonate in presence of triethylamine in dichloromethane to provide (9R)-9-((triisopropylsilyl)oxy)-6,7,8,9-tetrahydro-5H-cyclohepta[b]pyridin-5-one compound of formula-4.

7. The crystalline form-N of Rimegepant base compound of formula-1, which is characterized by:
i) Its powder X-ray diffractogram having peaks at about 5.2, 10.5, 15.5 16.6, 17.9, 19.8, 20.2, 24.9, 26.1 and 31.7±0.2 degrees 2-theta.
ii) Its powder X-ray diffraction pattern as shown in FIG. 1.

8. A process for the preparation of crystalline Form-N of Rimegepant base compound of formula-1, comprising of:
a) Treating the compound of formula-7 with a mixture of isopropanol/water and neutralized with a base to provide (5S,6S,9R)-5-amino-6-(2,3-difluorophenyl)-6,7,8,9-tetrahydro-5H-cyclo hepta[b]pyridin-9-ol compound of formula-8,
b) reacting the compound of formula-8 in-situ with 1-(1-(1H-imidazole-1-carbonyl) piperidine-4-yl)-1H-imidazo[4,5-b]pyridin-2 (3H)-one compound of formula-9 in presence of potassium tert-butoxide solution in THF to provide (5S,6S,9R)-5-amino-6-(2,3-difluorophenyl)-6,7,8,9-tetrahydro-5H-cyclohepta [b]pyridin-9-yl 4-(2-oxo-2,3-dihydro-1H-imidazo[4,5-b]pyridin-1-yl)-1-piperidine-1-carboxylate compound of formula-1,
c) purifying the compound with a suitable solvent to provide crystalline Form-N of Rimegepant compound of formula-1.

9. The process as claimed in claim-8, comprising of:
a) Treating the compound of formula-7 with a mixture of isopropanol/water and neutralized with sodium hydroxide to provide (5S,6S,9R)-5-amino-6-(2,3-difluorophenyl)-6,7,8,9-tetrahydro-5H-cyclohepta[b]pyridin-9-ol compound of formula-8,
b) reacting the compound of formula-8 in-situ with 1-(1-(1H-imidazole-1-carbonyl) piperidine-4-yl)-1H-imidazo[4,5-b]pyridin-2 (3H)-one compound of formula-9 in presence of potassium tert-butoxide solution in THF to provide (5S,6S,9R)-5-amino-6-(2,3-difluorophenyl)-6,7,8,9-tetrahydro-5H-cyclohepta [b]pyridin-9-yl 4-(2-oxo-2,3-dihydro-1H-imidazo[4,5-b]pyridin-1-yl)-1-piperidine-1-carboxylate compound of formula-1,
c) purifying the compound with methanol to provide crystalline Form-N of Rimegepant compound of formula-1.

10. The crystalline form-N of Rimegepant base of formula-1 is useful in the preparation of high pure Rimegepant hemisulfate sesquihydrate of formula-1a.
